SEQUENCE LISTING (58 sequences)

SEQ ID NO: 1 - protein, 29 aa, Artificial
  source: /note=“Description of artificial sequence IgSP-tPA pre-propeptide”
  SIGNAL (1)..(19): murine immunoglobulin signal peptide
  PROPEP (20)..(29): human tissue plasminogen activator propeptide
MKCSWVIFFLMAVVTGVNSSQEIHARFRR

SEQ ID NO: 2 - protein, 35 aa, Homo sapiens
MDAMKRGLCCVLLLCGAVFVSPSQEIHARFRRGAR

SEQ ID NO: 3 - protein, 19 aa, Mus musculus
  SIGNAL (1)..(19)
MKCSWVIFFLMAVVTGVNS

SEQ ID NO: 4 - protein, 26 aa, Homo sapiens
  SIGNAL (1)..(26)
MATGSRTSLLLAFGLLCLPWLQEGSA

SEQ ID NO: 5 - protein, 17 aa, Homo sapiens
  SIGNAL (1)..(17)
MLLLLLLLGLRLQLSLG

SEQ ID NO: 6 - DNA, 87 bp, Artificial
  source: /note=“Description of artificial sequence nucleic acid encoding IgSP-tPA pre-propeptide”
atgaagtgcagctgggtgatcttcttcctgatggccgtggtgaccggcgtgaattccagccaggagatccacgccaggttccgcaga

SEQ ID NO: 7 - DNA, 105 bp, Homo sapiens
atggacgccatgaagcgcggcctgtgctgcgtgctgctgctgtgcggcgccgtgttcgtgagccccagccaggagatccacgccaggttccgcagaggcgccaga

SEQ ID NO: 8 - DNA, 57 bp, Mus musculus
atgaagtgcagctgggtgatcttcttcctgatggccgtggtgaccggcgtgaattcc

SEQ ID NO: 9 - protein, 161 aa, Homo sapiens
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIEN

SEQ ID NO: 10 - protein, 239 aa, Homo sapiens [sequence omitted]

SEQ ID NO: 11 - DNA, 34 bp, Artificial
  source: /note=“Description of artificial sequence primer”
tgctctagagcgtcacccctagagtcgagctgtg

SEQ ID NO: 12 - DNA, 59 bp, Artificial
  source: /note=“Description of artificial sequence primer”
ggcgttgagcggccgcggttcatgacgctagcaccgaattcacccctgtaaccactgcc

SEQ ID NO: 13 - DNA, 51 bp, Artificial
  source: /note=“Description of artificial sequence primer”
cgcagatctggtctcgcatgaagtgcagctgggtgatcttcttcctgatgg

SEQ ID NO: 14 - DNA, 43 bp, Artificial
  source: /note=“Description of artificial sequence primer”
ccgtggtgaccggcgtgaattccgacagcgtgtgccctcaggg

SEQ ID NO: 15 - DNA, 52 bp, Artificial
  source: /note=“Description of artificial sequence primer”
caagtacatccaccctcagaacaacagcatctgctgcaccaagtgccacaag

SEQ ID NO: 16 - DNA, 26 bp, Artificial
  source: /note=“Description of artificial sequence primer”
ggcacctacctgtacaacgactgccc

SEQ ID NO: 17 - DNA, 51 bp, Artificial
  source: /note=“Description of artificial sequence primer”
gggcagtcgttgtacaggtaggtgcccttgtggcacttggtgcagcagatg

SEQ ID NO: 18 - DNA, 48 bp, Artificial
  source: /note=“Description of artificial sequence primer”
ctgttgttctgagggtggatgtacttgccctgagggcacacgctgtcg

SEQ ID NO: 19 - DNA, 48 bp, Artificial
  source: /note=“Description of artificial sequence primer”
gaattcacgccggtcaccacggccatcaggaagaagatcacccagctg

SEQ ID NO: 20 - DNA, 25 bp, Artificial
  source: /note=“Description of artificial sequence primer”
cacttcatgcgagaccagatctgcg

SEQ ID NO: 21 - DNA, 47 bp, Artificial
  source: /note=“Description of artificial sequence primer”
ccacaccatggccaccggcagccgcaccagcctgctgctggccttcg

SEQ ID NO: 22 - DNA, 50 bp, Artificial
  source: /note=“Description of artificial sequence primer”
gcctgctgtgcctgccctggctgcaggagggcagcgccgacagcgtgtgc

SEQ ID NO: 23 - DNA, 53 bp, Artificial
  source: /note=“Description of artificial sequence primer”
cctcagggcaagtacatccaccctcagaacaacagcatctgctgcaccaagtg

SEQ ID NO: 24 - DNA, 52 bp, Artificial
  source: /note=“Description of artificial sequence primer”
ccacaagggcacctacctgtacaacgactgccctggccctggccaggacacc

SEQ ID NO: 25 - DNA, 52 bp, Artificial
  source: /note=“Description of artificial sequence primer”
gactgccgcgagtgcgagagcggcagcttcaccgccagcgagaaccacctgc

SEQ ID NO: 26 - DNA, 55 bp, Artificial
  source: /note=“Description of artificial sequence primer”
gccactgcctgagctgcagcaagtgccgcaaggagatgggccaggtggagatcag

SEQ ID NO: 27 - DNA, 25 bp, Artificial
  source: /note=“Description of artificial sequence primer”
cagctgcaccgtggaccgcgacacc

SEQ ID NO: 28 - DNA, 53 bp, Artificial
  source: /note=“Description of artificial sequence primer”
ggtgtcgcggtccacggtgcagctgctgatctccacctggcccatctccttgc

SEQ ID NO: 29 - DNA, 53 bp, Artificial
  source: /note=“Description of artificial sequence primer”
ggcacttgctgcagctcaggcagtggcgcaggtggttctcgctggcggtgaag

SEQ ID NO: 30 - DNA, 53 bp, Artificial
  source: /note=“Description of artificial sequence primer”
ctgccgctctcgcactcgcggcagtcggtgtcctggccagggccagggcagtc

SEQ ID NO: 31 - DNA, 56 bp, Artificial
  source: /note=“Description of artificial sequence primer”
gttgtacaggtaggtgcccttgtggcacttggtgcagcagatgctgttgttctgag

SEQ ID NO: 32 - DNA, 49 bp, Artificial
  source: /note=“Description of artificial sequence primer”
ggtggatgtacttgccctgagggcacacgctgtcggcgctgccctcctg

SEQ ID NO: 33 - DNA, 47 bp, Artificial
  source: /note=“Description of artificial sequence primer”
cagccagggcaggcacagcaggccgaaggccagcagcaggctggtgc

SEQ ID NO: 34 - DNA, 23 bp, Artificial
  source: /note=“Description of artificial sequence primer”
ggctgccggtggccatggtgtgg

SEQ ID NO: 35 - DNA, 49 bp, Artificial
  source: /note=“Description of artificial sequence primer”
gctcgagatctggtctcgcatgctgctgctgctgctgctgctgggcctg

SEQ ID NO: 36 - DNA, 41 bp, Artificial
  source: /note=“Description of artificial sequence primer”
aggctccagctctccctgggcgacagcgtgtgccctcaggg

SEQ ID NO: 37 - DNA, 43 bp, Artificial
  source: /note=“Description of artificial sequence primer”
gcccagggagagctggagcctcaggcccagcagcagcagcagc

SEQ ID NO: 38 - DNA, 52 bp, Artificial; identical to SEQ ID NO: 15
  source: /note=“Description of artificial sequence primer”

SEQ ID NO: 39 - DNA, 26 bp, Artificial
  source: /note=“Description of artificial sequence primer”
ggcacctacctgtacaacgactgccc

SEQ ID NO: 40 - DNA, 51 bp, Artificial; identical to SEQ ID NO: 17
  source: /note=“Description of artificial sequence primer”

SEQ ID NO: 41 - DNA, 47 bp, Artificial
  source: /note=“Description of artificial sequence primer”
ctgttgttctgagggtggatgtacttgccctgagggcacacgctgtc

SEQ ID NO: 42 - DNA, 51 bp, Artificial; identical to SEQ ID NO: 13
  source: /note=“Description of artificial sequence primer”

SEQ ID NO: 43 - DNA, 52 bp, Artificial; identical to SEQ ID NO: 15
  source: /note=“Description of artificial sequence primer”

SEQ ID NO: 44 - DNA, 26 bp, Artificial
  source: /note=“Description of artificial sequence primer”
ggcacctacctgtacaacgactgccc

SEQ ID NO: 45 - DNA, 51 bp, Artificial; identical to SEQ ID NO: 17
  source: /note=“Description of artificial sequence primer”

SEQ ID NO: 46 - DNA, 47 bp, Artificial; identical to SEQ ID NO: 41
  source: /note=“Description of artificial sequence primer”

SEQ ID NO: 47 - DNA, 40 bp, Artificial
  source: /note=“Description of artificial sequence primer”
tctggcgcctctgcggaacctggcgtggatctcctggctg

SEQ ID NO: 48 - DNA, 48 bp, Artificial; identical to SEQ ID NO: 19
  source: /note=“Description of artificial sequence primer”

SEQ ID NO: 49 - DNA, 43 bp, Artificial
  source: /note=“Description of artificial sequence primer”
ccgtggtgaccggcgtgaattccagccaggagatccacgccag

SEQ ID NO: 50 - DNA, 486 bp, Homo sapiens
gacagcgtgtgccctcagggcaagtacatccaccctcagaacaacagcatctgctgcaccaagtgccacaagggcacctacctgtacaacgactgccctggccctggccaggacaccgactgccgcgagtgcgagagcggcagcttcaccgccagcgagaaccacctgcgccactgcctgagctgcagcaagtgccgcaaggagatgggccaggtggagatcagcagctgcaccgtggaccgcgacaccgtgtgcggctgccgcaagaaccagtaccgccactactggagcgagaacctgttccagtgcttcaactgcagcctgtgcctgaacggcaccgtgcacctgagctgccaggagaagcagaacaccgtgtgcacctgccacgccggcttcttcctgcgcgagaacgagtgcgtgagctgcagcaactgcaagaagagcctggagtgcaccaagctgtgcctgcctcagatcgagaactaa

SEQ ID NO: 51 - DNA, 19 bp, Artificial
  source: /note=“Description of artificial sequence primer”
ctagcaaaataggctgtcc

SEQ ID NO: 52 - DNA, 34 bp, Artificial
  source: /note=“Description of artificial sequence primer”
gttccgcagagacagcgtgtgccctcagggcaag

SEQ ID NO: 53 - DNA, 31 bp, Artificial
  source: /note=“Description of artificial sequence primer”
cacgctgtctctgcggaacctggcgtggatc

SEQ ID NO: 54 - DNA, 53 bp, Artificial; identical to SEQ ID NO: 30
  source: /note=“Description of artificial sequence primer”

SEQ ID NO: 55 - DNA, 53 bp, Artificial
  source: /note=“Description of artificial sequence primer”
ctgctgctgtgcggcgccgtgttcgtgagccccagccaggagatccacgccag

SEQ ID NO: 56 - DNA, 53 bp, Artificial; identical to SEQ ID NO: 30
  source: /note=“Description of artificial sequence primer”

SEQ ID NO: 57 - DNA, 57 bp, Artificial
  source: /note=“Description of artificial sequence primer”
gggctcacgaacacggcgccgcacagcagcagcacgcagcacaggccgcgcttcatg

SEQ ID NO: 58 - DNA, 30 bp, Artificial
  source: /note=“Description of artificial sequence primer”
gccaccatggacgccatgaagcgcggcctg

(19) United States
(12) Patent Application Publication
(10) Pub. No.: US 2007/0141666 A1
(43) Pub. Date: Jun. 21, 2007

(54) Leader sequences for use in production of proteins

(75) Inventors: Philippe Dupraz, Crissier (CH); Michel Kobr, Echandens (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Applied Research Systems ARS Holding N.V., Curacao (NL)

(21) Appl. No.: 10/573,369

(22) PCT Filed: Sep. 24, 2004
(86) PCT No.: PCT/EP04/52302

§ 371 Date: Dec. 26, 2006

Related U.S. Application Data
(60) Provisional application No. 60/506,939, filed on Sep. 29, 2003.

(30) Foreign Application Priority Data
Sep. 26, 2003 (EP) 03103580.1

(51) Int. Cl. C07K 14/47; C07H 21/04; C12P 21/06

(52) U.S. Cl. 435/69.1; 435/320.1; 435/325; 530/324; 536/23.5

(57) ABSTRACT

This invention encompasses novel leader sequences for production of proteins. More specifically, the invention relates to DNA constructs encoding leader sequences comprising an immunoglobulin signal peptide fused to a tissue-type plasminogen activator propeptide, and to DNA constructs encoding leader sequences comprising a truncated human tissue-type plasminogen activator propeptide. The invention further relates to the use of these DNA constructs for producing proteins in mammalian cells.

### FIELD OF THE INVENTION

This invention relates to leader sequences for production of proteins. More specifically, the invention relates to DNA constructs encoding leader sequences comprising an immunoglobulin signal peptide fused to a tissue-type plasminogen activator propeptide. The invention further relates to the use of these DNA constructs for producing proteins in mammalian cells.

### BACKGROUND

1. Processing of Protein Precursors

Secreted proteins are expressed initially inside the cell in a precursor form containing a leader sequence ensuring entry into the secretory pathway. Such leader sequences, named signal peptides, direct the expressed product across the membrane of the endoplasmic reticulum (ER). Signal peptides are generally cleaved off by signal peptidases during translocation to the ER. Once entered in the secretory pathway, the protein is transported to the Golgi apparatus. From the Golgi the protein can follow different routes that lead to compartments such as the cell vacuole or the cell membrane, or it can be routed out of the cell to be secreted to the external medium (Pfeffer and Rothman (1987) Ann. Rev. Biochem. 56:829-852).

For Industrial production of a secreted protein, the protein to be produced needs to be secreted efficiently from the host cell or the host organism. The signal peptide may be, e.g., the native signal peptide of the protein to be produced, a heterologous signal peptide, or a hybrid of native and heterologous signal peptide. Numerous signal peptides are used for production of secreted proteins. One of them Is a murine immunoglobulin signal peptide (IgSP, EMBL Accession No. M13331). IgSP was first identified in 1983 by Loh et al. (Cell. 33:85-93). IgSP is known to give a good expression in mammalian cells. For example. EP patent No. 0382762 discloses a method of producing horseradish peroxidase by constructing a fusion polypeptide between IgSP and horseradish peroxidase.

However, several problems are encountered with the use of currently known signal peptides. One problem often encountered when producing a human protein from a non-human host cell or organism is that the native signal peptide does not ensure efficient translocation and/or cleavage of the signal peptide. This leads to low rates of protein secretion and/or to secretion of mature proteins that display N-terminal extensions due to an incorrect cleavage of the signal peptide. Thus the choice of the signal peptide is of great Importance for industrial production of a protein.

In addition of leader sequences directing the secretion of the protein, a precursor form can comprise supplemental leader sequences that are cleaved during maturation. These supplemental leader peptides, named propeptides, usually follow the signal peptide. Virtually all peptide hormones, numerous bloactive proteins (for example, growth factors, receptors and cell-adhesion molecules), and many bacterial toxins and viral envelope glycoproteins comprise a propeptide that is post-translationally excised to generate the mature and biologically active protein (Seidah and Chretien (1999) Brain Res. 848:45-62).

Propeptides are cleaved off by enzymes named proprotein convertases. Mammalian proprotein convertases include, e.g., the subtilisin convertases PCSK1, PCSK2 and furin. Furin is ubiquitously expressed and located in the trans-Golgi network. Furin proteolytically activates large numbers of proproteins substrates in secretory pathway compartments. (Thomas (2002) Nat Rev Mol Cell Biol. 3:753-766). More specifically, furin localizes to the Trans Golgi Network—a late Golgi structure that is responsible for sorting secretory pathway proteins to their final destinations, including the cell surface, endosomes, lysosomes and secretory granules. The site that furin cleaves has been extensively studied. The cleavage site is positioned after the carboxyl-terminal arginine of the consensus sequence R-X-L/R-R, wherein X may represent any amino acid (Nakayama (1997) Biochem. J 327:625-635). The cleavage efficiency is increased when X is a lysine, a valine, an isoleucine or an alanine (Watanabe et al (1992) J Biol. Chem. 267:8270-8274).

2. The Tissue-type Plasminogen Activator Precursor

The human tissue-type plasminogen activator precursor (tPA, Swiss Prot Acession No. P00750) is synthesized as a precursor form of 562 amino acids comprising a leader sequence of 35 amino acids. This leader sequence comprises a signal peptide of 23 amino acids followed by a propeptide of 12 amino acids.

Köhne et al. (1999, J. Cell. Biochem. 75 :446-461) showed that the tPA leader sequence of 35 amino acids was able to rescue intracellular transport of a chimeric Tumor Necrosis Factor Receptor—immunoglobulin protein (TNFR-Ig) in which all N-linked glycosylation sites had been deleted. In 1999, Etcheverry et al. reported that a leader sequence of 13 amino acids, which comprised the last amino acid of the signal peptide and the entire propeptide of tPA, was able to enhance secretion of a TNFR-Ig fusion when inserted between the TNFR native signal peptide and the TNFR-Ig polypeptide (Etcheverry et al., ESACT meeting, abstract O1.07/P1.02).

Thus protein processing is a fundamental process for efficient protein secretion, and the choice of the leader sequence is a critical step when producing a secreted polypeptide. In many cases, the leader sequence leads to a low level of secretion or no secretion at all, or to an incorrect or incomplete proteolytic processing. It is therefore the object of the present invention to provide leader sequences that ensure a more efficient secretion and/or processing of polypeptides.

### SUMMARY OF THE INVENTION

The present Invention is based on the finding that a leader sequence comprising an immunoglobulin signal peptide fused to a tissue-type plasminogen activator propeptide allows a more efficient secretion and processing of proteins of interest than other known leader sequences. In addition, it has been found that a leader sequence comprising a truncated form of the human tPA propeptide, wherein the carboxyl-terminal extremity of the tPA propeptide consists of amino acids Arg-Xaa-Arg-Arg, allows an efficient secretion and processing of proteins of interest.

Therefore, a first aspect of the invention relates to a DNA construct comprising a sequence encoding an IgSP-tPA pre-propeptide comprising an immunoglobulin signal peptide fused to a tPA propeptide.

A second aspect relates to A DNA construct comprising a sequence encoding a human tissue-type plasminogen activator propeptide (tPA) wherein the carboxyl-terminal extremity of said tPA propeptide consists of amino acids Arg-Xaa-Arg-Arg

A third aspect relates to a host cell transformed with a DNA construct according to the invention.

A fourth aspect relates to a process for the production of a polypeptide of interest comprising the step of transfecting a host cell with a DNA construct in accordance with the invention.

A fifth aspect relates to a process for the production of a polypeptide of interest comprising the step of culturing a host cell of the invention.

A sixth aspect relates to the use of a DNA construct in accordance with the invention for producing a polypeptide of interest.

A seventh aspect relates to a fusion polypeptide encoded by a DNA construct in accordance with the invention.

### BRIEF DESCRIPTION OF THE FIGURES

FIG. 1 shows an alignment between an IgSP pre-propeptide in accordance with the invention (SEQ ID NO: 1) and the native tPA pre-propeptide (SEQ ID NO: 2).

FIG. 2 shows a scheme of the pGL3-GH-TBP-1 380 vector used to construct the different signal peptide fused to the TBPI protein.

FIG. 3 shows a scheme of the pEF1-GH-TBP-1403 vector used to express the leader peptides-TBPI fusion proteins in transient transfection assays.

FIG. 4 shows the amount of TBPI protein detected in supernatant versus cytoplasm of cells transfected with the indicated constructs. Lane GH_SP: TBPI fused to the Growth hormone signal peptide; Lane SEAP_SP: TBPI fused to the secreted alkaline phosphatase signal peptide; Lane IgSP: TBPI fused to the murine immunogl obulin IgG μ-heavy chain signal peptide; Lane IgSP-tPA: TBPI fused to an IgSP pre-propeptide in accordance with the invention.

FIG. 5 corresponds to a scheme of the CMV-UbB-LUC-1433 vector used to express the leader peptides-TBPI fusion proteins in stable transfection assays.

FIG. 6 shows the amont of TBPI protein detected in the supematant of pools of clones transfected with IgSP-tPA or with tPA-tPA pre-propeptides fused to TBPI. Pools were maintained eitheir in puromycin and neomycin co-selection (neo/puro) or in puromycin minus Hypoxantine-Tymidine co-selection (HT/puro). Open box and dark box represent two different pulses of 48 hrs at 37° C. in medium with 10% FCS. Stripped or squared box represent two pulses of 48 hrs at 32° C. In serum-free medium.

### BRIEF DESCRIPTION OF THE SEQUENCES OF THE SEQUENCE LISTING

SEQ ID NO: 1 corresponds to the protein sequence of an IgSP-tPA pre-propeptide in accordance with the invention.

SEQ ID NO: 2 corresponds to the protein sequence of the human tPA pre-propeptide.

SEQ ID NO: 3 corresponds to the protein sequence of the murine IgG μ-heavy chain signal peptide.

SEQ ID NO: 4 corresponds to the protein sequence of the human growth hormone signal peptide.

SEQ ID NO: 5 corresponds to the protein sequence of the human secreted alkaline phosphatase signal peptide.

SEQ ID NO: 6 corresponds to the nucleic sequence of an IgSP-tPA pre-propeptide in accordance with the invention.

SEQ ID NO: 7 corresponds to the nucleic sequence of the human tPA pre-propeptide.

SEQ ID NO: 8 corresponds to the nucleic sequence of the murine IgG μ-heavy chain signal peptide.

SEQ ID NO: 9 corresponds to the protein sequence of the soluble portion of the TNF receptor p55.

SEQ ID NO: 10 corresponds to the protein sequence of mature interferon gamma receptor chain.

SEQ ID Nos. 11 to 20 correspond to primers used to construct and amplify the Ig μ-heavy chain signal peptides.

SEQ ID NO: 21 to 34 correspond to primers used to construct and amplify the human growth hormone signal peptide.

Seq ID NO: 35 to 41 correspond to primers used to construct and amplify the human secreted alkaline phosphatase signal peptide.

Seq ID NO: 42 to 49 correspond to primers used to construct and amplify the IgSP-tPA pre-propeptide in accordance with this invention.

Seq ID NO: 50: correspond to the nucleic sequence of the soluble extracellular portion of the p55 Tumor necrosis factor.

Seq ID NO: 51 to 54 correspond to primers used to introduce a deletion of three amino-acids into the IgSP-tPA and tPA pre-propeptides in accordance with this invention.

Seq ID NO: 55 to 58 correspond to primers used to generate the tPA-pre-propeptide.

### DETAILED DESCRIPTION OF THE INVENTION

The present invention stems from the finding that leader sequences comprising an immunoglobulin signal peptide fused to a tissue-type plasminogen activator propeptide allow a more efficient secretion and processing of proteins than other known leader sequences. As shown in examples 2 and 3, leader sequences of the present invention are at least 2 fold more efficient in promoting secretion of proteins of interest than prior art leader sequences.

In addition, it has been found that a leader sequence comprising a truncated form of the human tPA propeptide, wherein the carboxyl-terminal extremity of the tPA propeptide consists of amino acids Arg-Xaa-Arg-Arg, allows an efficient secretion and processing of proteins of interest.

Accordingly, the present invention provides novel leader sequences comprising (i) an immunoglobulin signal peptide fused to a tissue-type plasminogen activator propeptide; or (ii) a tPA propeptide wherein its carboxyl-terminal extremity consists of amino acids Arg-Xaa-Arg-Arg. The use of these leader sequences for producing proteins of interest in mammalian cells is a further aspect of the present invention.

A first aspect of the present invention relates to a DNA construct comprising a sequence encoding an IgSP-tPA pre-propeptide comprising an immunoglobulin signal peptide fused to a tissue-type plasminogen activator propeptide.

As used herein, the term “signal peptide” refers to a leader sequence ensuring entry into the secretory pathway. As used herein, the term “propeptide” refers to a leader sequence that follows a signal peptide. As used herein, the term “pre-propeptide” refers to a leader sequence comprising a signal peptide and a propeptide. As used herein, the term “leader sequence” refers to a sequence located at the amino terminal end of the precursor form of a protein. Leader sequences are cleaved off during maturation.

As further used herein, the term “IgSP-tPA pre-propeptide” refers to a leader sequence according to the present invention that comprises an immunoglobulin signal peptide fused to a tissue-type plasminogen activator propeptide.

As shown in example 1, IgSP-tPA pre-propeptides ensures a more efficient secretion of the soluble portion of the TNF receptor p55 (TBPI) than various signal peptides fused directly to TBPI. Example 2 demonstrates that the IgSP-tPA pre-propeptide is more efficient in promoting TBPI secretion than a tPA pre-propeptide alone. Example 3 demonstrates that the IgSP-tPA pre-propeptide is more efficient in promoting secretion of the mature interferon gamma receptor chain (IFNAR) than the native IFNAR signal peptide. Accordingly, IgSP-tPA pre-propeptides ensure a more efficient secretion of polypeptides of interest than any known leader sequence.

Numerous immunoglobulin (Ig) signal peptides from different species are known and are all encompassed within the scope of the present invention. In a preferred embodiment, the Ig signal peptide is a murine immunoglobulin signal peptide. Preferably, the murine Ig signal peptide is a murine IgG μ-heavy chain signal peptide of SEQ ID NO: 3.

The DNA construct of the present invention comprises a sequence encoding a tissue-type plasminogen activator (tPA) propeptide of any origin. For example, the DNA construct can comprise a sequence encoding a tPA propeptide of human, murine, rat or bovin origin. Preferably, the DNA construct comprises a sequence encoding a tPA propeptide of human origin.

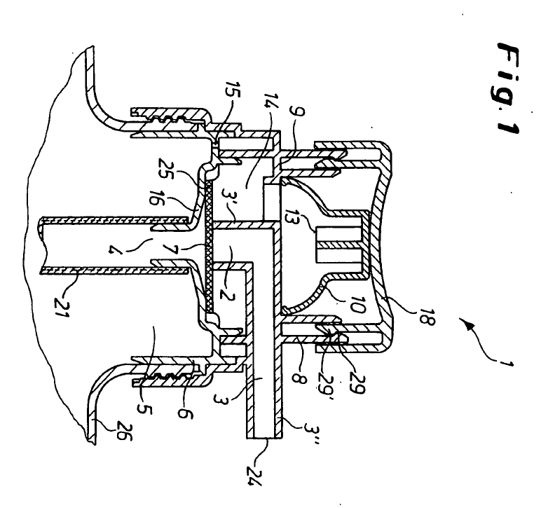

In a further embodiment, the DNA construct of the present invention comprises a sequence encoding a human tPA propeptide, the carboxyl-terminal extremity of said tPA propeptide consisting of amino acids Arg-Xaa-Arg-Arg. Such a propeptide corresponds to a truncated propeptide lacking the three carboxyl-terminal amino acids of the native human tPA propeptide (see FIG. 1). Preferably, the human tPA propeptide consists either of amino acids 24 to 32 of SEQ ID NO: 2 or of amino acids 23 to 32 of SEQ ID NO: 2.

In a preferred embodiment, the DNA construct of the present invention encodes a pre-propeptide comprising SEQ ID NO: 1. In a most preferred embodiment, the pre-propeptide consists of SEQ ID NO: 1.

The terms “comprising”, “consisting of”, or “consisting essentially of” have distinct meanings. However, each term may be substituted for another herein to change the scope of the invention.

As used herein, the expression “a polypeptide A fused to a polypeptide B” refers to a fusion polypeptide comprising the sequences of polypeptides A and B, wherein the sequence of polypeptide A is located at the amino-terminal extremity of the sequence of polypeptide B within said fusion polypeptide. The term “fused to”, as used herein, is not limited to a direct fusion of polypeptides. For example, the cloning strategy may lead to the presence of amino acids between polypeptides A and B. However, a direct fusion of polypeptides is preferred. Methods of constructing DNA constructs comprising sequences encoding fusion polypeptides are well known in the art. For example, methods described in examples 1 to 3 may be used.

Another preferred embodiment of the present invention relates to a DNA construct encoding a fusion polypeptide comprising an IGSP-tPA pre-propeptide fused to a polypeptide of interest.

In accordance with the present invention, the polypeptide of interest may be any polypeptide for which production is desired. For example, the polypeptide of interest may be, e.g., a naturally secreted protein, a normally cytoplasmic protein, a normally transmembrane protein, or a human or a humanized antibody. When the protein of interest is a normally cytoplasmic or a normally transmembrane protein, the protein has preferably been engineered in order to become soluble. The polypeptide of interest may be of any origin. Preferred polypeptides of interest are of human origin.

Preferably, the first amino acid of the polypeptide of interest is not an aliphatic hydrophobic amino acid. Should the first amino acid of the naturally occurring polypeptide of interest be an aliphatic hydrophobic amino acid, the protein of interest has preferably been engineered so that its first amino acid is not an aliphatic hydrophobic amino acid.

In preferred embodiments, the polypeptide of interest is selected from the group consisting of chorionic gonadotropin, follicle-stimulating hormone, lutropin-choriogonadotropic hormone, thyroid stimulating hormone, human growth hormone, interferons (e.g., interferon beta-1a, interferon beta-1b), interferon receptors (e.g., interferon gamma receptor), TNF receptors p55 and p75, interleukins (e.g., interleukin-2, interleukin-11), interleukin binding proteins (e.g., interleukin-18 binding protein), anti-CD11a antibodies, and muteins, fragments, soluble forms, functional derivatives, fusion proteins thereof.

Other preferred polypeptides of interest include, e.g., erythropoietin, granulocyte colony stimulating factor, granulocyte-macrophage colony-stimulating factor, pituitary peptide hormones, menopausal gonadotropin, insulin-like growth factors (e.g., somatomedin-C), keratinocyte growth factor, glial cell line-derived neurotrophic factor, thrombomodulin, basic fibroblast growth factor, insulin, Factor VIII, somatropin, bone morphogenetic protein-2, platelet-derived growth factor, hirudin, epoletin, recombinant LFA-3/IgG1 fusion protein, glucocerebrosidase, and muteins, fragments, soluble forms, functional derivatives, fusion proteins thereof.

A second aspect of the present invention is directed to a DNA construct comprising a sequence encoding a human tissue-type plasminogen activator propeptide characterized in that its carboxyl-terminal extremity consists of amino acids Arg-Xaa-Arg-Arg.

In a first embodiment, the tPA propeptide of the present invention is a human tPA propeptide consisting either of amino acids 24 to 32 of SEQ ID NO: 2 or of amino acids 23 to 32 of SEQ ID NO: 2.

In a second embodiment, the DNA construct comprising a tPA propeptide in accordance with the invention further comprises a signal sequence fused to said tPA propeptide.

Any signal peptide currently used in the art for promoting protein secretion may be used in the above embodiment. Such signal peptides include, e.g., the human growth hormone signal peptide (see, e.g., EP application 01 999 6 52.9), the secretion competent polypeptide disclosed in EP application 00 906 103.7, the human erythropoietin signal peptide, the human albumin signal peptde, the human secreted alkaline phosphatase signal peptide and the rotavirus VP7 glycoprotein signal peptide.

In a third embodiment, the DNA comprising a tPA propeptide in accordance with the invention encodes a fusion polypeptide comprising said tPA propeptide fused to a polypeptide of interest.

In a preferred embodiment, the DNA construct comprising a sequence encoding an IgSP-tPA pre-propeptide and/or a tPA propeptde in accordance with the invention is included in a vector.

The term “vector” refers to any carrier of exogenous DNA or RNA that is useful for transferring exogenous DNA to a host cell for replication and/or appropriate expression of the exogenous DNA by the host cell.

In a further preferred embodiment, the vector is an expression vector. An “expression vector” comprises appropriate signals that drive expression in host cells of a polynucleotide inserted In said vector. Preferably, the polynucleotides inserted in said vector encode a polypeptide of interest. The appropriate signals include various regulatory elements, such as enhancers and/or promoters from both viral and mammalian sources. Selectable markers for establishing permanent, stable cell clones expressing the products such as, e.g., a dominant drug selection, are generally included in the expression vectors of the invention, as they are elements that link expression of the drug s election markers to expression of the polypeptide.

In a further preferred embodiment, the vector is a vector for performing gene activation. The gene activation technology is a technology allowing the production of proteins of interest without introducing the gene or the cDNA of interest into the host cell (see e.g., EP patents Nos. 0 505 500 and 0 779 362). For example, the gene activation technology may bypass regulatory DNA sequences set in the “off position” with regulatory DNA sequences set in the “on position” in order to activate the gene of interest. A gene activation vector comprises appropriate signals that drive expression in host cells of a polynucleotide present in said host cell.

In a further preferred embodiment, the vector is a gene therapy vector. Expression vectors that may be used for gene therapy are well known in the art. Preferably, the gene therapy vector is a lentiviral derived vector, which has been shown to be very efficient in the transfer of genes, in particular within the CNS. Other well-established viral vectors, such as adenoviral derived vectors, may also be used according to the invention.

A third aspect of the invention relates to a host cell transformed with a DNA construct according to the invention. Many host cells are suitable in accordance with the present invention, such as primary or established cell lines from a wide variety of eukaryotes including plant and animal cells. Preferably, said host cell is an eukaryotic cell. Most preferably, said host cell is a mammalian cell.

For example, suitable host cells include CHO cells, COS cells, CV1 cells, mouse L cells, HT1080 cells, BHK-21 cells, HEK293 cells, NIH-3T3 cells, LM cells, YI cells, NSO and SP2/0 mouse hybridoma cells and the like, Namalwa cells, RPMI-8226 cells, Vero cells, WI-38 cells, MRC-5cells or other immortalized and/or transformed cells.

Preferably, the host cell is a CHO cell, and more preferably a CHO—S cell, described e.g. by Shotwell et al. (1982, J Biol. Chem. 257:2974-2980).

In a fourth aspect, the invention relates to a process for the production of a polypeptide of interest comprising the step of transfecting a host cell with a DNA construct according to the invention.

In a fifth aspect, the invention relates to a process for the production of a polypeptide of interest comprising the step of culturing a host cell in accordance with the invention.

Such processes according to the invention lead to secretion of the protein of interest, which may be harvested from the cell culture supernatant. Depending on the intended use, the cell synthetizing the polypeptide may be the product of the process according to the invention.

In a preferred embodiment, these processes further comprise the step of isolating the polypeptide of interest from the host cells. This step is particularly advantageous and easy to carry out since the protein of Interest may simply be isolated from the cell culture supernatant.

These processes may be used in transient, stable, episomal or viral expression systems. In a preferred embodiment, the transfection is stable transfection.

In a sixth aspect, the DNA construct according to the invention is used for producing a polypeptide of interest.

A seventh aspect of the invention relates to fusion polypeptides comprising an IgSP-tPA pre-propeptide and/or a tPA propeptide according to the invention fused to a polypeptide of interest.

Having now fully described this invention, it will be appreciated by those skilled in the art that the same can be performed within a wide range of equivalent parameters without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished patent application, issued patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

### Example 1

### Comparison Between the IgSP-tPA Pre-propeptide and the Human Growth Hormone Signal Peptide, the Secreted Alkaline Phosphatase Signal Peptide, the Murine Immunogobulin Signal Peptide

1.1. Constructions

1.1.1 IgSP

The murine IgG μ-heavy chain signal peptide of SEQ ID NO: 3 (IgSP) cloned as follows. Primers of Seq ID Nos. 13 to 20 were incubated with the T4 polynucleotide kinase (Stratagene) for 2 h 30 at 37° C., and heat inactivated at 75° C. for 10 min. The treated primers were ligated using cycle ligation with Pfu Ligase from Stratagene as recommended by the manufacturer in the following cycle conditions:

95° C. for 1 min;

40 cycles at 95° C. for 30″, 57° C. for 90″, 70° C. for 2 min

70° C. for 10 min.

The annealed oligos were then purified with QIAquick columns, and PCR amplified with PFU turbo using standard conditions with the primer SEQ ID No 13 and 17 under the following PCR conditions:

95° C. 5min

30 cycles of 95° C. for 45″, 70° C. for 45″; and

70° C. for 10 min.

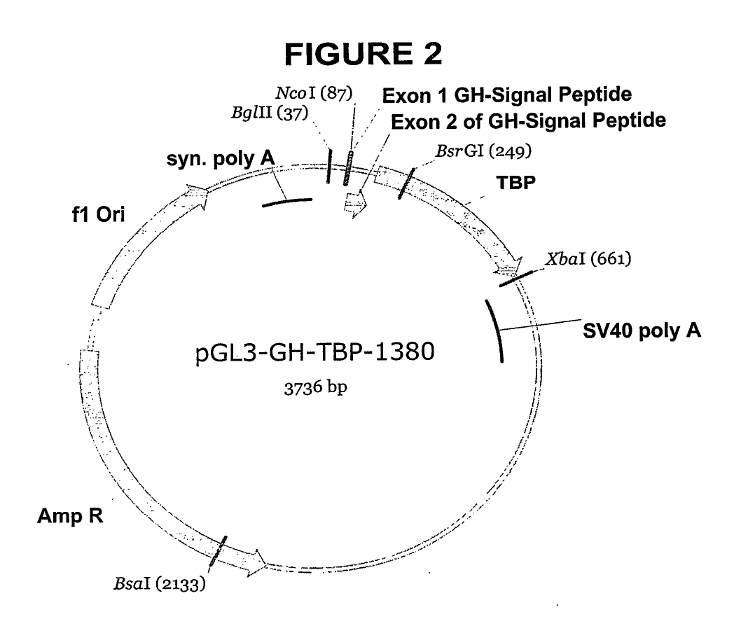

The PCR product was purified and digested with Bgl -Il and BsrGI and cloned into the pGL3-GH-TBPI-1380 vector (FIG. 2). The product was sequenced and was found to reflect the expected sequence.

1.1.2 GH_SP

The human growth hormone signal peptide of SEQ ID NO: 4 (GH_SP) was cloned as follows using primers of SEQ ID No. 21 to 34. The primers were annealed as described above. At the end of the annealing, the product was purified and re-amplified by PCR using the same conditions as above. The PCR product was re-amplified using primers of SEQ ID Nos. 21 and 34. The product was then cloned into the pGL3 -GH-TBPI380 vector at the BgI-II and BsrGI cloning sites.

1.1.3. SEAP_SP

The human secreted alkaline phosphatase signal peptide of SEQ ID NO: 5 (SEAP_SP) was cloned using primers of SEQ ID No. 35 to 41. The primers were annealed as described above. At the end of the annealing, the product was purified and re-amplified by PCR using the same conditions as above. The PCR product was re-amplified using primers of SEQ ID Nos. 35 and 40. The product was then cloned into the pGL3-GH-TBPI380 vector at the BgI-II and BsrGI cloning sites.

1.1.4. IgSP-tPA

The studied IgSP-tPA pre-propeptide comprised: (i) the murine IgG μ-heavy chain signal peptide fused to (ii) a truncated tPA propeptide that lacks the three carboxyl-terminal amino acids of the native tPA propeptide. This IgSP-tPA pre-propeptide is shown as SEQ ID NO: 1. This IgSP-tPA pre-propeptide was cloned as follows.

Primers of SEQ ID No. 42 to 49 were annealed as described above. At the end of the annealing, the product was purified and re-amplified by PCR using primers of SEQ ID 45 and 49. The purified PCR product was then cloned into the pGL3 -GH-TBPI380 vectors at the BgI-II and BsrGI cloning sites.

A recombinant PCR was performed for deleting three amino-acid (GAR) from the known tPA propeptide. The 5′ end of the construct was PCR amplified with primers of SEQ ID Nos. 51 and 52, and the 3′ end was PCR amplified with primers of SEQ ID Nos. 53 and 54. The full-length IgSP-tPA-TBPI construct was then PCR amplified with primers of SEQ ID Nos. 51 and 54 and the two recombinant products obtained from the previous PCR.

1.1.5. Fusion of the Above Leader Sequences to TBPI

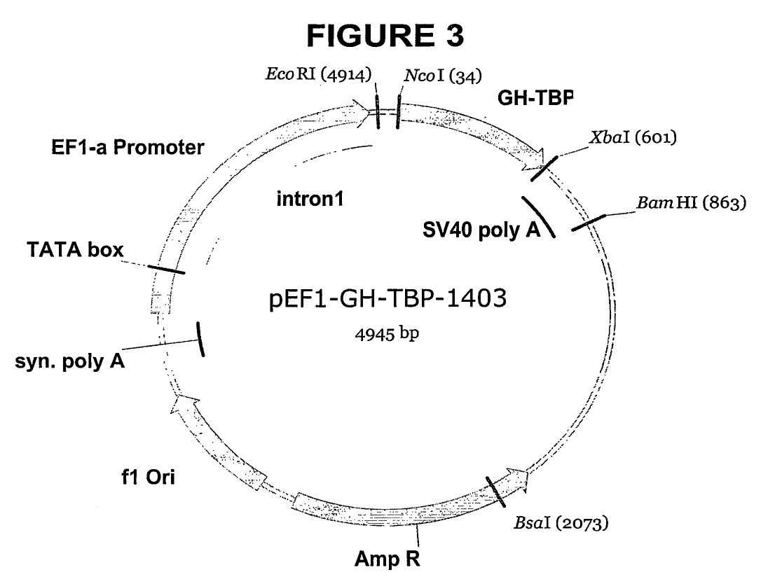

The above leader sequences were fused to the soluble portion of the TNF receptor p55 protein (TBPI, SEQ ID NO: 50). The pEF1-GH-TBPI-1403 vector (FIG. 3), which allows expression of TBPI under the Human Elongation Factor 1 (EF1) promoter, was digested by Nco-I and Xba-I. Fragments encoding the leader sequences were subcloned into pEF1-GH-TBPI-1403 as Nco-I or as Bsa-I-Xba-I fragments. The resulting IgSP-TBPI, GH_SP-TBPI SEAP_SP-TBPI and IgSP-tPA constructs were sequenced and were found to reflect the expected sequence.

1.2. Measurement of Protein Secretion

Each construct was transfected in CHO-DUKX-B11 cells using standard lipid mediated transfection as described in standard laboratory manuals (Maniatis et al., Molecular Cloning: A Laboratory Manual, 2^{nd} edition, Cold Spring Harbor Laboratory Press). 48 hrs after transfection, medium and cells were harvested. Cells were washed twice in PBS and the pellet was lysed on ice using 300 μl of Cytobuster buffer (Novagen) in the presence of a protease inhibitor cocktail (Roche). Cellular debries were spinned down by centrifugation at 16 000 g for 10 mn at 4° C. The supernatant was harvested and kept at −20° C. before being processed. The amount of TBPI released in the supematant or in intracellular compartment was analyzed by an ELISA. The relative expression of TBPI was measured and reported.

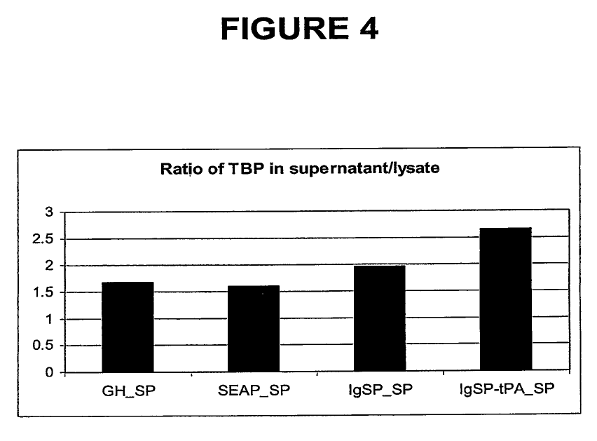

The results of the experiment are shown on FIG. 4. The IgSp-tPA signal propeptide is able to boost secretion of TBPI from cells as demonstrated by the increased amount of TBPI detected in the supematant versus the amount of TBPI detected in intrace lullar compartments. Thus the IgSP-tPA-TBPI construct, comprising TBPI fused to an IgSP-tPA propeptide, increases secretion of TBPI compared to the constructs corresponding to the TBPI protein fused to the IgSP signal peptide, to the secreted alkaline phosphatase signal peptide or to the growth hormone signal peptide.

Accordingly, the IgSP-tPA pre-propeptide ensures a more efficient secretion of TBPI than any other signal peptide fused directly to TBPI without propeptide.

### Example 2: Comparison between the lacSP-tPA pre-proieptide and the tPA pre-propeptide.

2.1. Constructions

2.1.1. IgSP-tPA-TBPI

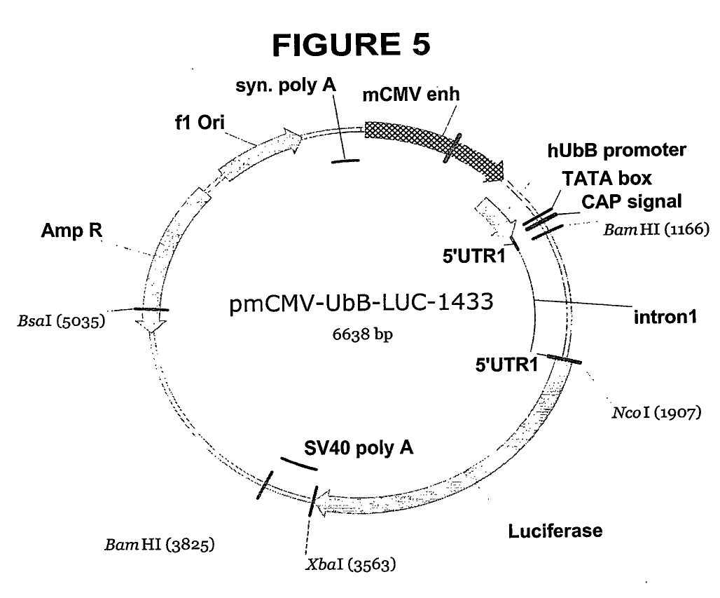

The IgSP-tPA-TBPI construct described in 1.1.5. was digested by Xba-I. The fragment comprising IgSP-tPA-TBPI was cloned into the pmCMV-UbB-LUC-1433 expression vector (FIG. 5) digested by Nco-I and Xba-I.

2.1.2. tPA-TBPI

A tPA pre-propeptide of SEQ ID NO: 2 comprising: (i) the tPA signal peptide and (ii) a truncated tPA propeptide that lacks the three carboxyl-terminal amino acids of the native tPA propeptide was generated as follows.

The human tPA pre-propeptide was cloned using the IgSP-tPA-TBPI construct as a template. A first PCR was performed with primers of SEQ ID No 55 and 56 in order to amplify the tPA propeptide and the 5′ end of TBPI. In a second PCR, t he PCR product from the first step was extended by re-amplification with primers of SEQ ID Nos. 57, 58 and 56. The PCR product was then cloned into the pGL3-GH-TBPI-1380 vector (FIG. 2) digested with BgI-II and BsrGI. A recombinant PCR was performed as described in 1.1.4. with primers of SEQ ID Nos. 51 to 54 in order to introduce an Internal deletion of three amino-acid (GAR) into the known tPA propeptide.

The resulting tPA-TBPI construct was digested by a Bsa-I and Xba-I. The fragment comprising tPA-TBPI was cloned into the pmCMV-UbB-LUC-1433 expression vector digested with Nco-I and Xba-I.

2.2. Measurement of Protein Secretion

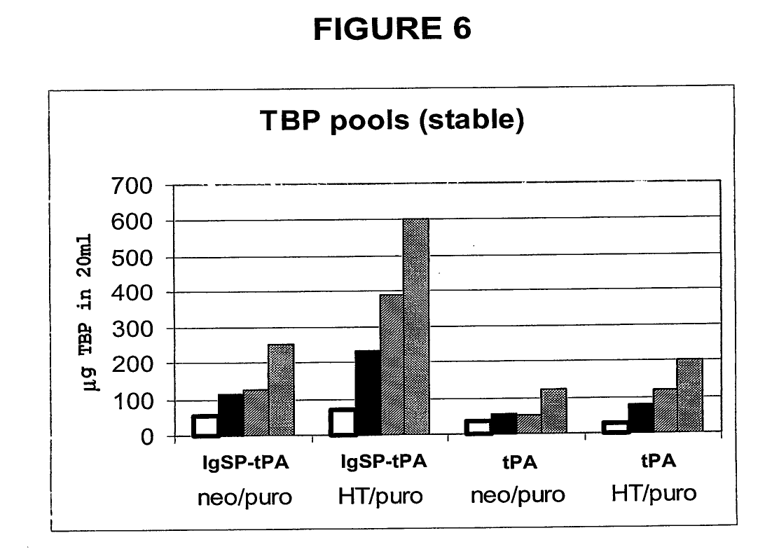

CHO cells were transfected using lipofectamine with the IgSP-tPA-TBPI and the tPA-TBPI constructs. In one series of experiments, the TBPI expression vector was co-transfected with SV40neo and puro vectors for selection. In another series, the TBPI expression vector co-transfected with SV40dhfr and SV40 puro vectors. Pools of stable expressing clones representing at least 100 clones were expanded in different selection medium (600 μg.ml⁻¹ neomycin, 6 μg.ml⁻¹ puromycin, or HT+6 μg.ml⁻¹ puromycin). Pools were split and cells seeded either in FCS-containing or in serum-free medium. The media were harvested after 48 hrs, and the amount of TBPI released In the supematant was determined using an ELISA. The result of this experiment is shown in FIG. 6. Each box represents a pool. Open boxes and dark boxes represent two different pulses in 10% FCS-containing medium at 37° C. Striped or squared box represent two different pulses in serum-free medium at 32° C. The initial number of seeded cells and the pulse periods were similar in each experiment.

FIG. 6 shows that in all conditions that were studied, pools of IgSP-tPA-TBPI expressing cells had higher titers of TBPI than pools of tPA-TBPI expressing cells. The results clearly indicate that the IgSP-tPA construct is at least two fold better than tPA construct in terms of quantity of secreted protein that is produced.

Accordingly, the novel combination of the tPA propeptide with the IgSP signal peptide is more efficient at promoting secretion of proteins than the tPA pre-propeptide.

In addition, the sequence of the N-terminal extremity of the TBPI protein secreted from IgSP-tPA-TBPI expressing cells was determined by N-terminal sequencing using Edman degradation. It was found that 100% of the proteins had been cleaved after the last arginine residue of the tPA. Thus the IgSP-tPA pre-propeptide ensures an efficient and reliable processing of polypeptides.

### Example 3

### Comparison between the IgSP-tPA Pre-Propeptide and the Interferon Gamma Receptor Signal Peptide for Production of Interferon Gamma.

3.1. Constructs

The IgSP pre-propeptide was fused to a mature interferon gamma receptor chain protein (IFNAR) and cloned into (i) the mCMV-UbB-LUC-1433 vector (FIG. 3); or (ii) a vector comprising the promoter of the mCMV-IE2 gene described in EP application 03 100 617.4.

A full-length IFNAR, comprising the native signal peptide, was cloned into (i) the mCMV-UbB-LUC-1433 vector; or (ii) the expression vector comprising the promoter of the mCMV-IE2 gene described in EP application 03 100 617.4.

3.2. Measurement of Protein Secretion

3.2.1. Protocol

Constructs were transfected into CHO cells using standard lipid mediated transfection protocols. The secreted proteins were harvested after 48 hrs. A specific Elisa test was used to quantify the amount of IFNAR secreted in the supernatant. The transfections were normalized with a luciferase construct co-transfected with the IFNAR vector. A standard luciferase assay was used as described in standard laboratory manuals (Maniatis et al., Molecular Cloning: A Laboratory Manual, 2^{nd} edition, Cold Spring Harbor Laboratory Press).

3.2.1. Constructs Comprising the CMV Vector

The IgSP-tPA pre-propeptide was about 2.3 fold more efficient in promoting secretion of the IFNAR protein in the supernatant than the native IFNAR signal peptide.

3.2.2 Constructs Comprising the Promoter of the mCMV-IE2 Gene.

The IgSP-tPA pre-propeptide was about 4.3 fold more efficient in promoting secretion of the IFNAR protein in the supematant than the native IFNAR signal peptide.

### REFERENCES

Etcheverry et al., ESACT meeting, abstract O1.07/P1.02
Köhne et al (1999) J. Cell. Blochem. 75:446-461
Loh et al. (1983) Cell. 33:85-93
Nakayama (1997) Biochem. J 327:625-635
Pfeffer and Rothman (1987) Ann. Rev. Biochem. 56:829-852
Seidah and Chretien (1999) Brain Res. 848:45-62
Shotwell et al. (1982) J Biol. Chem. 257:2974-2980
Thomas (2002) Nat Rev Mol Cell Biol. 3:753-766
Watanabe et al (1992) J Biol. Chem. 267:8270-8274

1-23. (canceled)

24. A composition of matter comprising:
a) a DNA construct comprising a sequence encoding an IGSP-tPA pre-propeptide comprising an immunoglobulin signal peptide (IgSP) fused to a tissue-type plasminogen activator (tPA) propeptide;
b) a DNA construct comprising a sequence encoding an IgSP-tPA pre-propeptide comprising a murine immunoglobulin signal peptide (IgSP) fused to a tissue-type plasminogen activator (tPA) propeptide;
c) a DNA construct comprising a sequence encoding an IgSP-tPA pre-propeptide comprising the murine immunoglobulin signal peptide (IgSP) of SEQ ID NO: 3 fused to a tissue-type plasminogen activator (tPA) propeptide;
d) a DNA construct comprising a sequence encoding a fusion protein, said fusion protein comprising an IgSP-tPA pre-propeptide fused to a polypeptide of interest and said IgSP-tPA pre-propeptide comprising an immunoglobulin signal peptide (IgSP) fused to a tissue-type plasminogen activator (tPA) propeptide;
e) a DNA construct comprising a sequence encoding a human tissue-type plasminogen activator propeptide (tPA) wherein the carboxyl-terminal extremity of said tPA propeptide consists of amino acids Arg-Xaa-Arg-Arg;
f) a DNA construct comprising a sequence encoding an IgSP-tPA pre-propeptide comprising an immunoglobulin signal peptide (IgSP) fused to a tissue-type plasminogen activator (tPA) propeptide;
g) a vector comprising a DNA construct comprising a sequence encoding an IgSP-tPA pre-propeptide comprising a murine immunoglobulin signal peptide (IgSP) fused to a tissue-type plasminogen activator (tPA) propeptide;
h) a vector comprising a DNA construct comprising a sequence encoding an IgSP-tPA pre-propeptide comprising the murine immunoglobulin signal peptide (IgSP) of SEQ ID NO: 3 fused to a tissue-type plasminogen activator (tPA) propeptide;
i) a vector comprising a DNA construct comprising a sequence encoding a fusion protein, said fusion protein comprising an IgSP-tPA pre-propeptide fused to a polypeptide of interest and said IgSP-tPA pre-propeptide comprising an immunoglobulin signal peptide (IgSP) fused to a tissue-type plasminogen activator (tPA) propeptide;
j) a vector comprising a DNA construct comprising a sequence encoding a human tissue-type plasminogen activator propeptide (tPA) wherein the carboxyl-terminal extremity of said tPA propeptide consists of amino acids Arg-Xaa-Arg-Arg; or
k) a host cell transformed with the DNA construct or vector comprising:
1) a DNA construct comprising a sequence encoding an IgSP-tPA pre-propeptide comprising an immunoglobulin signal peptide (IgSP) fused to a tissue-type plasminogen activator (tPA) propeptide;
2) a DNA construct comprising a sequence encoding an IgSP-tPA pre-propeptide comprising a murine immunoglobulin signal peptide (IgSP) fused to a tissue-type plasminogen activator (tPA) propeptide;
3) a DNA construct comprising a sequence encoding an IgSP-tPA pre-propeptide comprising the murine immunoglobulin signal peptide (IgSP) of SEQ ID NO: 3 fused to a tissue-type plasminogen activator (tPA) propeptide;
4) a DNA construct comprising a sequence encoding a fusion protein, said fusion protein comprising an IgSP-tPA pre-propeptide fused to a polypeptide of interest and said IgSP-tPA pre-propeptide comprising an immunoglobulin signal peptide (IgSP) fused to a tissue-type plasminogen activator (tPA) propeptide;
5) a DNA construct comprising a sequence encoding a human tissue-type plasminogen activator propeptide (tPA) wherein the carboxyl-terminal extremity of said tPA propeptide consists of amino acids Arg-Xaa-Arg-Arg;
6) a vector comprising a DNA construct comprising a sequence encoding an IgSP-tPA pre-propeptide comprising an immunoglobulin signal peptide (IgSP) fused to a tissue-type plasminogen activator (tPA) propeptide;
7) a vector comprising a DNA construct comprising a sequence encoding an IgSP-tPA pre-propeptide comprising a murine immunoglobulin signal peptide (IgSP) fused to a tissue-type plasminogen activator (tPA) propeptide;
8) a vector comprising a DNA construct comprising a sequence encoding an IgSP-tPA pre-propeptide comprising the murine immunoglobulin signal peptide (IgSP) of SEQ ID NO: 3 fused to a tissue-type plasminogen activator (tPA) propeptide;
9) a vector comprising a DNA construct comprising a sequence encoding a fusion protein, said fusion protein comprising an IgSP-tPA pre-propeptide fused to a polypeptide of interest and said IgSP-tPA pre-propeptide comprising an immunoglobulin signal peptide (IgSP) fused to a tissue-type plasminogen activator (tPA) propeptide; or
10) a vector comprising a DNA construct comprising a sequence encoding a human tissue-type plasminogen activator propeptide (tpA) wherein the carboxyl-terminal extremity of said tPA propeptide consists of amino acids Arg-Xaa-Arg-Arg.

25. The composition of matter according to claim 24, wherein the tPA propeptide encoded by said DNA construct is a human tPA propeptide, the carboxyl-terminal extremity of said tPA propeptide consisting of amino acids Arg-Xaa-Arg-Arg.

26. The composition of matter according to claim 25, wherein said tPA propeptide consists of amino acids 23 to 32 of SEQ ID NO: 2.

27. The composition of matter according to claim 24, wherein the pre-propeptide encoded by said genetic construct comprises SEQ ID NO: 1.

28. The composition of matter according to claim 25, wherein the pre-propeptide encoded by said genetic construct comprises SEQ ID NO: 1.

29. The composition of matter according to claim 26, wherein the pre-propeptide encoded by said genetic construct comprises SEQ ID NO: 1.

30. The composition of matter according to claim 24, wherein said vector is an expression vector.

31. The composition of matter according to claim 24, wherein said vector is a vector for performing gene activation.

32. The composition of matter according to claim 24, wherein the host cell comprises a DNA construct encoding human tPA propeptide, the carboxyl-terminal extremity of said tPA propeptide consisting of amino acids Arg-Xaa-Arg-Arg.

33. The composition of matter according to claim 24, wherein the host cell comprises a DNA construct encoding a tPA propeptide consisting of amino acids 23 to 32 of SEQ ID NO: 2.

34. The composition of matter according to claim 24, wherein the host cell comprises a DNA construct and the pre-propeptide encoded by said DNA construct comprises SEQ ID NO: 1.

35. The composition of matter according to claim 24, wherein said host cell is selected from the group consisting of a CHO cell, a COS cell, a CV1 cell, a mouse L cell, a HT1080 cell, a BHK cell, a HEK293 cell, a NIH-3T3 cell, a LM cell and a Y1 cell, NSO and SP2/0 mouse hybridoma and the like, Namalwa, RPMI-8226, Vero, WI-38, and MRC-5.

36. The composition of matter according to 35, wherein said cell is a CHO cell.

37. A process for the production of a polypeptide of interest comprising the step of transfecting a host cell with a DNA construct or vector comprising:
a) a DNA construct comprising a sequence encoding an IgSP-tPA pre-propeptide comprising an immunoglobulin signal peptide (IgSP) fused to a tissue-type plasminogen activator (tPA) propeptide;
b) a DNA construct comprising a sequence encoding an IgSP-tPA pre-propeptide comprising a murine immunoglobulin signal peptide (IgSP) fused to a tissue-type plasminogen activator (tPA) propeptide;
c) a DNA construct comprising a sequence encoding an IgSP-tPA pre-propeptide comprising the murine immunoglobulin signal peptide (IgSP) of SEQ ID NO: 3 fused to a tissue-type plasminogen activator (tPA) propeptide;
d) a DNA construct comprising a sequence encoding a fusion protein, said fusion protein comprising an IgSP-tPA pre-propeptide fused to a polypeptide of interest and said IgSP-tPA pre-propeptide comprising an immunoglobulin signal peptide (IgSP) fused to a tissue-type plasminogen activator (tPA) propeptide;
e) a DNA construct comprising a sequence encoding a human tissue-type plasminogen activator propeptide (tPA) wherein the carboxyl-terminal extremity of said tPA propeptide consists of amino acids Arg-Xaa-Arg-Arg;
f) a vector comprising a DNA construct comprising a sequence encoding an IgSP-tPA pre-propeptide comprising an immunoglobulin signal peptide (IgSP) fused to a tissue-type plasminogen activator (tPA) propeptide;
g) a vector comprising a DNA construct comprising a sequence encoding an IgSP-tPA pre-propeptide comprising a murine immunoglobulin signal peptide (IgSP) fused to a tissue-type plasminogen activator (tPA) propeptide;
h) a vector comprising a DNA construct comprising a sequence encoding an IgSP-tPA pre-propeptide comprising the murine immunoglobulin signal peptide (IgSP) of SEQ ID NO: 3 fused to a tissue-type plasminogen activator (tPA) propeptide;
i) a vector comprising a DNA construct comprising a sequence encoding a fusion protein, said fusion protein comprising an IgSP-tPA pre-propeptide fused to a polypeptide of interest and said IgSP-tPA pre-propeptide comprising an immunoglobulin signal peptide (IgSP) fused to a tissue-type plasminogen activator (tPA) propeptide; or
j) a vector comprising a DNA construct comprising a sequence encoding a human tissue-type plasminogen activator propeptide (tPA) wherein the carboxyl-terminal extremity of said tPA propeptide consists of amino acids Arg-Xaa-Arg-Arg;
to produce a polypeptide of interest.

38. The process according to claim 37, firther comprising the step of culturing the host cell.

39. The process according to claim 37, further comprising the step of isolating the polypeptide of interest from said host cells.

40. The process according to claim 37, wherein the transfection is stable transfection.

41. A polypeptide comprising:
a) an IgSP-tPA pre-propeptide comprising an immunoglobulin signal peptide (IgSP) fused to a tissue-type plasminogen activator (tPA) propeptide;
b) an IgSP-tPA pre-propeptide comprising a murine immunoglobulin signal peptide fused to a tissue-type plasminogen activator (tPA) propeptide;
c) an IgSP-tPA pre-propeptide comprising a murine immunoglobulin signal peptide comprising SEQ ID NO: 3 fused to a tissue-type plasminogen activator (tPA) propeptide;
d) an IgSP-tPA pre-propeptide comprising an immunoglobulin signal peptide (IgSP) fused to a tissue-type plasminogen activator (tPA) propeptide, wherein said tPA propeptide is a human tPA propeptide, the carboxyl-terminal extremity of said tPA propeptide consisting of amino acids Arg-Xaa-Arg-Arg;
e) an IgSP-tPA pre-propeptide comprising a murine immunoglobulin signal peptide fused to a tissue-type plasminogen activator (tPA) propeptide, wherein said tPA propeptide is a human tPA propeptide, the carboxyl-terminal extremity of said tPA propeptide consisting of amino acids Arg-Xaa-Arg-Arg;
f) an IgSP-tPA pre-propeptide comprising a murine immunoglobulin signal peptide comprising SEQ ID NO: 3 fused to a tissue-type plasminogen activator (tPA) propeptide, wherein said tPA propeptide is a human tPA propeptide and the carboxyl-terminal extremity of said tPA propeptide consisting of amino acids Arg-Xaa-Arg-Arg;
g) a human tissue-type plasminogen activator propeptide (tPA) wherein the carboxyl-terminal extremity of said tPA propeptide consists of amino acids Arg-Xaa-Arg-Arg;
h) a tPA propeptide consisting of amino acids 23 to 32 of SEQ ID NO: 2;
i) a human tissue-type plasminogen activator propeptide (tPA), wherein the carboxyl-terminal extremity of said tPA propeptide consists of amino acids Arg-Xaa-Arg-Arg or a tPA propeptide consists of amino acids 23 to 32 of SEQ ID NO: 2, each further comprising a signal sequence fused to said tPA propeptide; or
j) a polypeptide of interest fused to: 1) a human tissue-type plasminogen activator propeptide (tPA) with a carboxyl-terminal extremity consisting of amino acids Arg-Xaa-Arg-Arg fused to a signal sequence or 2) a tPA propeptide consisting of amino acids 23 to 32 of SEQ ID NO: 2 fused to a signal sequence.

42. The polypeptide according to claim 41, wherein said tPA propeptide consists of amino acids 23 to 32 of SEQ ID NO: 2.

43. The polypeptide according to claim 41, wherein said pre-propeptide comprises SEQ ID NO: 1.

44. The polypeptide according to claim 41, wherein said IgSP-tPA pre-propeptide fused to a polypeptide of interest.
